(19) United States
(12) Patent Application Publication
(10) Pub. No.: US 2021/0398672 A1
(43) Pub. Date: Dec. 23, 2021

(54) Big Data-Driven Personalized Management of Chronic Pain

(71) Applicant: Northwestern University, Evanston, IL (US)

(72) Inventors: Apkar Vania Apkarian, Chicago, IL (US); Marwan Baliki, Chicago, IL (US); Etienne Vachon-Presseau, Montreal (CA); Lejian Huang, Evanston, IL (US); Thomas J. Schnitzer, Chicago, IL (US)

(21) Appl. No.: 17/289,813

(22) PCT Filed: Oct. 28, 2019
(86) PCT No.: PCT/US19/58348

§ 371 Date: Apr. 29, 2021

Related U.S. Application Data
(60) Provisional application No. 62/751,780, filed on Oct. 29, 2018.

(51) Int. Cl. G16H 50/20; G16H 10/60; G16H 50/70; G16H 50/30; G16H 20/10

(52) U.S. Cl. CPC G16H 50/20; G16H 10/60; G16H 20/10; G16H 50/30; G16H 50/70

(57) ABSTRACT

Systems as described herein can include diagnosing and treating patients with chronic conditions, such as chronic back pain. A transition to chronic pain can include brain adaptations that can carve the state of chronic pain. Additionally, pain characteristics, pain related disability, and responses to treatment can be at least partially determined by psychological factors and/or personality properties. Personalized management recommendations can be generated for individual chronic pain patients using an infrastructure and associated methodology monitors chronic pain patients and gathers a large amount of group data (e.g., phenotyping participants at various levels of depth: behavior, psychology, brain anatomy and function, genetics, etc.) and machine learning methods to generate individualized treatment recommendations that are updated and retooled based on the group data analyses and based on the specific subjects responses.

### CROSS-REFERENCE TO RELATED APPLICATIONS

This application claims priority to U.S. Provisional Patent Application No. 62/751,780, titled “Big Data-Driven Personalized Management of Chronic Pain” and filed Oct. 29, 2018, the disclosure of which is hereby incorporated by reference in its entirety

### FEDERAL FUNDING STATEMENT

This invention was made with government support under AT007987, NS035115, and DE022746, each awarded by the National Institutes of Health. The government has certain rights in the invention.

### FIELD OF THE DISCLOSURE

This disclosure relates generally to improved pain management and, more particularly, to big data-driven personalized management of patients with chronic pain.

### BACKGROUND

Pain is associated with negative motions and is highly salient, enabling an organism to escape or protect an injured body part and thereby enhance its chance for survival. However, when pain becomes chronic and the subject must live with the constant pain for many years, the pain becomes maladaptive and modifies the subject's outlook on everyday experiences and future expectations by changing physiological and psychological processes underlying pain perception and pain-related behavior. Brain elements are involved in such processes.

Chronic pain impacts approximately 10% of adults and can occur in the absence of identifiable external stimuli. Chronic pain diminishes quality of life and increases anxiety and depression. Chronic pain can also be associated with cognitive, chemical, and/or morphologic abnormalities. However, there remains a lack of knowledge regarding brain elements involved in such conditions.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Systems as described herein can include diagnosing and treating patients with chronic conditions, such as chronic back pain. A transition to chronic pain can include brain adaptations that can carve the state of chronic pain. Additionally, pain characteristics, pain related disability, and responses to treatment can be at least partially determined by psychological factors and/or personality properties. Personalized management recommendations can be generated for individual chronic pain patients using an infrastructure and associated methodology monitors chronic pain patients and gathers a large amount of group data (e.g., phenotyping participants at various levels of depth: behavior, psychology, brain anatomy and function, genetics, etc.) and machine learning methods to generate individualized treatment recommendations that are updated and retooled based on the group data analyses and based on the specific subject's responses. A plurality of chronic pain types can be treated using a combination of gene, brain, and personality information to model the chronic pain and generate individual treatment options when correlated against the particular patient's responses.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1A shows an example methodology to generate personalized management recommendations for chronic pain patients in accordance with at least one aspect of the disclosure;

FIG. 1B illustrates a process to predict patient pain medication response in accordance with at least one aspect of the disclosure;

FIG. 1C illustrates a multi-layered artificial neural network in accordance with at least one aspect of the disclosure;

FIG. 2 shows a block diagram of an example of data collection and assessment in accordance with at least one aspect of the disclosure;

FIG. 3 illustrates a data-driven personalized management system in accordance with at least one aspect of the disclosure;

FIGS. 4A-4C depict a signature model predicting placebo propensity from brain and personality characteristics in accordance with at least one aspect of the disclosure;

FIGS. 5A-5E depict a perceptron that predicts future placebo pill response magnitude and categorical outcomes in accordance with at least one aspect of the disclosure;

FIG. 6 shows an example extent of information shared between two brain regions in accordance with at least one aspect of the disclosure;

FIGS. 7A-7C show predictability of drug treatment response based on brain function in accordance with at least one aspect of the disclosure;

FIG. 8 shows an example smartphone application tracking individual subject features in accordance with at least one aspect of the disclosure;

FIG. 9 shows an example system in accordance with at least one aspect of the disclosure; and

FIG. 10 is a block diagram of an example processor platform in accordance with at least one aspect of the disclosure.

### DETAILED DESCRIPTION

In the following detailed description, reference is made to the accompanying drawings that form a part hereof, and in which is shown by way of illustration specific examples that can be practiced. These examples are described in sufficient detail to enable one skilled in the art to practice the subject matter, and it is to be understood that other examples can be utilized and that logical, mechanical, electrical and other changes can be made without departing from the scope of the subject matter of this disclosure. The following detailed description is, therefore, provided to describe an exemplary implementation and not to be taken as limiting on the scope of the subject matter described in this disclosure. Certain features from different aspects of the following description can be combined to form yet new aspects of the subject matter discussed below.

Brain properties contribute to a risk of developing chronic pain. Additionally, a transition to chronic pain involves brain adaptations that can carve the state of chronic pain. Additionally, pain characteristics, pain related disability, and responses to treatment can be at least partially determined by psychological factors and/or personality properties. Although a biopsychosocial theoretical construct is actively applied in clinical management of chronic pain (e.g., multidisciplinary biopsychosocial rehabilitation for chronic low back pain: Cochrane systematic review and meta-analysis, etc.), component properties that form multidisciplinary biopsychosocial rehabilitation, such as neurobiological processes that determine the chronic pain psychology, remain unknown. Certain examples address this issue by determining whether psychological factors associated with chronic pain are identifiable and whether there are neurobiological underpinnings that can be quantified and reliably linked to the psychology of chronic pain.

About 20% of world population suffers from chronic pain. The large majority of such patients are not satisfied with their pain management. Such patients continue to suffer from high levels of pain, infrequent access to medical oversight, haphazard regiments and medications and procedures imposed on them with little objective evidence for the specific recommendation. Depending on the type of clinic any such patient visits, the treatments and therapies recommended can be vastly different. The ineffective and mostly randomly assigned therapies negatively impact the patients (e.g., through continued suffering, etc.), cost a huge amount in health care waste, and overall negatively impact on society through lost productivity and quality of life to a very large proportion of society worldwide.

Certain examples provide methods, systems, data collection and analysis tools, etc., to generate personalized management recommendations for individual chronic pain patients. Certain examples provide an infrastructure and associated methodology that uses internet monitoring of chronic pain patients and gathers a large amount of group data (e.g., phenotyping participants at various levels of depth: behavior, psychology, brain anatomy and function, genetics, etc.) and machine learning methods to generate individualized treatment recommendations that are updated and retooled based on the group data analyses and based on the specific subject's responses.

For example, chronic pain patients are asked a set of questions and brain and genetic data are gathered. Based on these answers and corresponding answers from a given participant, a score is calculated regarding adequacy of ongoing management of chronic pain for the particular individual. Participants are tracked over time using various measures, and their state of pain is updated, and alternatives are then suggested and tracked. Overall, this methodology relies on already existing information and new data collected in participants to diminish pain for each participant, using customized, semi-supervised, analyses.

Thus, a plurality of chronic pain types can be treated using a combination of gene, brain, and personality information to model the chronic pain and generate individual treatment options when correlated against the particular patient's responses. Certain examples provide an application to execute on smartphone and/or other computing device to aggregate and process data from a plurality of participants to calculate an optimized or otherwise improved treatment regimen for each chronic pain patient for which information is available.

By combining multi-dimensional properties of chronic pain patients (e.g., pain, affect, personality, behavior, with brain anatomy and physiology, with genetics, and blood inflammatory markers, etc.) and tracking pain, mood and medication or therapy use, certain examples generate individualized prediction of optimum/improved therapy regimens for a limited time window. The regimen/time window can be updated based on a subject's response and based on a continued model comparison between the subject and population subgroups, for example. However, it should be noted that any of a variety of patients with any of a variety of chronic disorders such as, but not limited to, chronic negative mood disorders, such as PTSD, depression, and anxiety, can be diagnosed and treated using any of the systems and processes described herein.

A data collection and analysis platform integrated with smartphone technology provides accurate prediction of optimal therapy for a given chronic pain patient, for a finite time period, based on past data collected in the subject, in relation to similar data collected in a larger cohort of subjects suffering from a similar type of chronic pain. Such information has utility in diminishing suffering with chronic pain, across all types of chronic pain.

In certain examples, the smartphone tool is an integrated, coherent, and comprehensive tool that is semi-autonomous, as a machine learning component undergoes continued oversight and adjustment of the types of learning rules incorporated, based on both observations collected from individuals as well as computer simulations used to reduce error between prediction and actual observations, for example. Additionally, clinical oversight can vet outcomes for consistency with clinical knowledge and provide feedback to the model.

In certain examples, subject input data collected is layered over various dimensions. For each dimension, data can be available at various depths of phenotyping. New entries to the system must provide a minimum set of information (most superficial) and then can continue to expand on this minimal set by opting to add to their information base across dimensions and depths, for example.

In certain examples, personality, brain anatomy, brain function, in combination with genetic information can predict risk for chronic pain. Also, personality and brain properties can identify subjects who are prone to respond to placebo. In addition, subjects who respond to duloxetine for pain relief are predictable from brain functional properties. Taken together, chronic pain management can be optimized based on tracking such information across a large group of participants and this information can be used for individualized optimization of pain management.

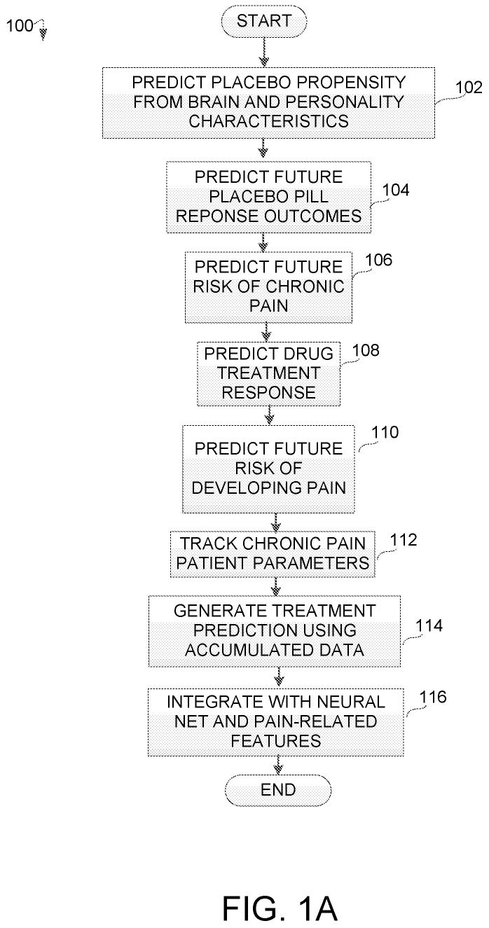

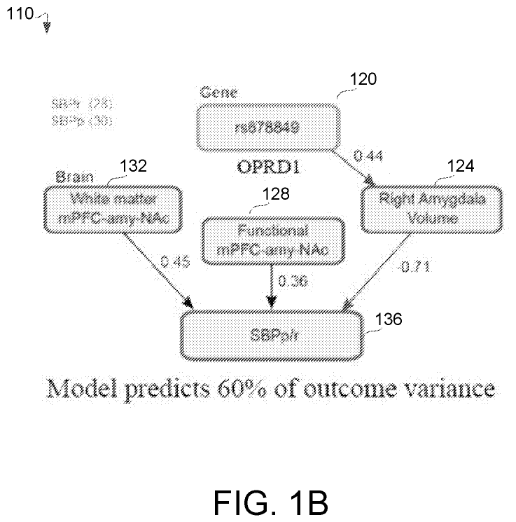

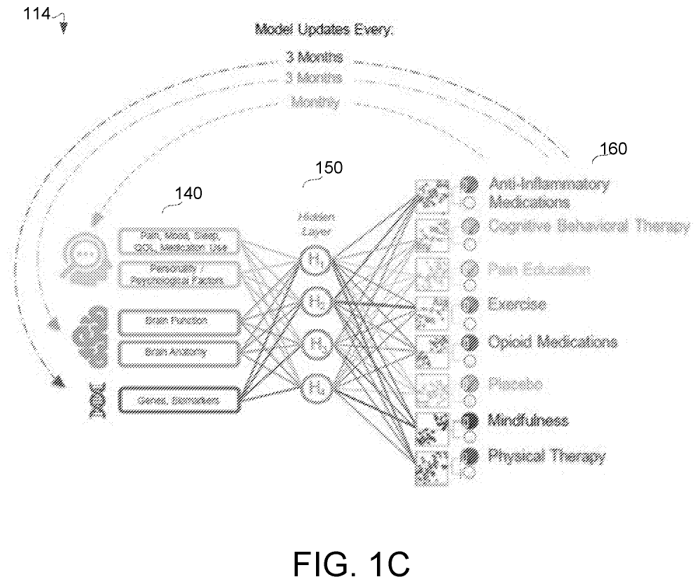

FIG. 1A shows an example methodology 100 followed to generate personalized management recommendations for chronic pain patients. At block 102, placebo propensity from brain and personality characteristics is predicted via machine learning. For example, receiver operating characteristics (ROC) curves are computed using a support vector machine (SVM) classifier for each functional, anatomical, and personality predictor. At block 104, future placebo pull response outcomes are predicted using a four-layered perceptron, wherein the specific areas of interest relevant to the placebo response are specific to the following: functional networks, anatomical predictors, sleep, personality traits, and components of the personality network.

At block 106, a future risk of chronic pain is predicted using ROC curves to estimate probability based on brain region information sharing, where focus is placed directly on the medical prefrontal cortex (mPFC) and nucleus accumbens (NAc). At block 108, prediction of the drug treatment response is obtained. For example, a patient response to pain medication duloxetine can be used such that duloxetine (and/or other serotonin and norepinephrine reuptake inhibitor (SNRI), selective serotonin reuptake inhibitor (SSRI), etc.) responders are identified as having a minimal predicted placebo and ≥20% empirical analgesia. Specifically, a focus on the right parahippocampal gyrus (r-PHG) degree counts is relevant in identifying future duloxetine response. At block 110, a future risk of developing pain is predicted.

FIG. 1B further illustrates a process taken to predict patient pain medication response using a combination of brain anatomy, functional connectivity, and gene expression identifiers, with the model predicting 60% of the outcome variance. For example, a model focusing on the expression of gene rs678849, shown at 120, brain anatomy at regions of the right amygdala 124 and white matter 132, and functional connectivity as identified by mPFC-amy-NAc, is combined to yield a prediction 136 of the future risk of chronic pain.

At block 112, chronic pain patient parameters are tracked over time. For example, this can be accomplished using a smart-phone application, with primary parameters of interest being pain level, mood, sleep, quality of life, and mobility. Patient-specific recommendations can be initiated in the process in a timely manner (e.g., every 6-12 weeks, etc.).

At block 114, accumulated data are used to generate treatment predictions. Specifically, a multi-layered artificial neural network is applied, as shown in FIG. 1C. Treatment generation is dependent upon multiple factors specific to participant characteristics obtained via phenotyping (e.g., demographics, personality, brain function, anatomy, genes). Specifically, the neural network integrates specific patient characteristics 140, processes the information 150, and generates an optimal treatment prediction 160. As shown in the example of FIG. 1C, given characteristics of a participant (e.g., phenotyping at different depths of knowledge: from demographics and personality, to brain function and anatomy, to genes, etc.), the neural net integrates time varying pain properties with deeper information and correlates these properties with treatment types used to identify correspondences between subject properties and optimal treatment options. Multiple architectures can be implemented to compete with each other to determine the best results. In time and based on performance, these networks are pruned down and rendered more automated.

At block 116, the data generated at block 114 is integrated with information already collected, which includes pain-related features. Data collection is continuously performed in a large group of participants, with revisions and corrections performed via clinical oversight to improve model stability and prediction, also improving the neural net learning rate. Thus, data is modeled as it is collected to accommodate ongoing data collection paired with dynamic improvement of the neural network learning rate, for example.

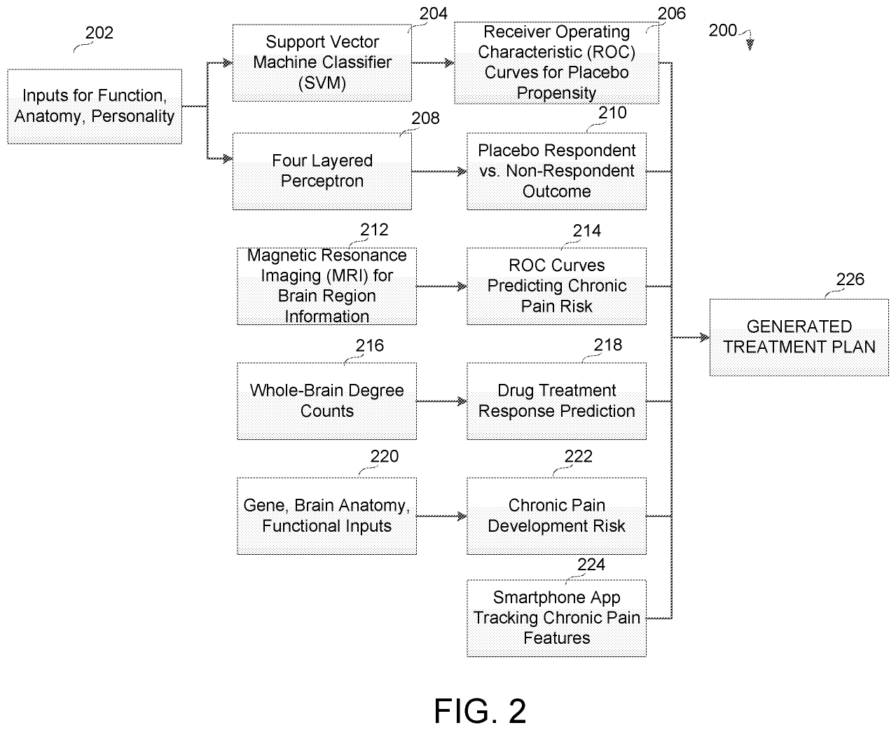

FIG. 2 shows a block diagram 200 as an example of a systematic approach to data collection and assessment that leads to a final generated treatment plan 226. Inputs for patient-specific function, anatomy, and personality 202 are used to initiate generation of receiver operating characteristic (ROC) curves 206 for placebo propensity and determination of whether a patient is or is not a responded to placebo-based medications, as shown at block 210. The ROC curves are generated via a support vector machine (SVM) classifier via input from 202, where these inputs include the following: MFG-PAG: middle frontal gyms-periaqueductal grey; MFG-rACC: middle frontal gyrus-rostral anterior cingulate cortex; MFG-M1: middle frontal gyrus-precentral gyrus; Limbic-R/L: limbic rightward asymmetry; Cort-thick: Cortical thickness in superior frontal gyrus; ERQ-s: Emotional Regulation suppression; MAIA-Emo: Multidimensional Assessment of Interoceptive Awareness emotional awareness; MAIA-NW: Multidimensional Assessment of Interoceptive Awareness not-worrying; PCS-Rumi: Pain Catastrophizing scale rumination; PCS-Help: Pain Catastrophizing scale helplessness. A 4-layered perceptron 208 receives 13 inputs to predict placebo pill response, with categorical outcomes for 3 output measures and 2 treatment timepoints, for example. The perceptron model is trained 100 times prior to determining grouping accuracy. To determine a probability 214 of chronic pain risk on the future, information 212 from brain regions mPFC and NAc are obtained. Additionally, drug treatment response prediction 218 is generated via whole-brain degree counts 216, specific to patients who are duloxetine responders.

In one example, results indicate that the right parahippocampal gyrus (r-PHG) is the region of interest, given that r-PHG degree count correlated with the difference between empirical analgesia and predicted placebo response for visual analog scale (VAS) (e.g., p=0.048) and Western Ontario & McMaster Universities Osteoarthritis Index (WOMAC) (e.g., p=0.033) outcomes, for a total of 20 patients. Additional patient-specific inputs 220 are provided to assess chronic pain development risk, as described in FIG. 1B. Finally, chronic pain features are tracked on a timely basis (e.g., every 6-12 weeks), with all of the information obtained permitting a final output 226 in the form of a generated treatment plan personalized to each patient, for example.

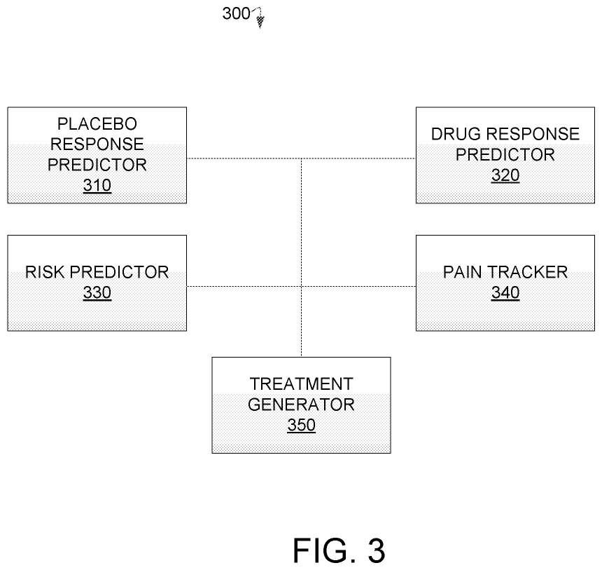

In certain examples, the system 200 can be implemented using a data-driven personalized management system 300 such as shown in FIG. 3. The example system 300 includes a placebo response predictor 310, a drug response predictor 320, a risk predictor 330, a pain tracker 340, and a treatment generator 350.

For example, the placebo response predictor 310 includes a machine learning model to predict placebo propensity from brain and personality characteristics. For example, receiver operating characteristics (ROC) curves are computed using a support vector machine (SVM) classifier for each functional, anatomical, and personality predictor. Future placebo pull response outcomes are predicted using a four-layered perceptron, wherein the specific areas of interest relevant to the placebo response are specific to the following: functional networks, anatomical predictors, sleep, personality traits, and components of the personality network. The example drug response predictor 320 generates a prediction of drug treatment response by the patient. For example, using the patient response to pain medication, a future response of the patient to drug treatment can be predicted. The example risk predictor 330 predicts a future risk of developing chronic pain. For example, a future risk of chronic pain is predicted using ROC curves to estimate probability based on brain region information sharing.

The example pain tracker 340 tracks patient chronic pain parameters over time. For example, the pain tracker 340 can include and/or be implemented as a software application, such as a smartphone application, etc., with primary parameters of interest being pain level, mood, sleep, quality of life, and mobility. Patient-specific recommendations can be initiated in the process in a timely manner (e.g., every 6-12 weeks, etc.).

The example treatment generator 350 uses accumulated data to generate treatment predictions. For example, a multi-layered artificial neural network is applied, such as shown in FIG. 1C. Treatment generation can be dependent upon multiple factors specific to participant characteristics obtained via phenotyping (e.g., demographics, personality, brain function, anatomy, genes). For example, the neural network integrates specific patient characteristics 140, processes the information 150, and generates an optimal treatment prediction 160. Data continues to be collected and aggregated with prior collected data to provide feedback to adjust the neural network, model(s), etc.

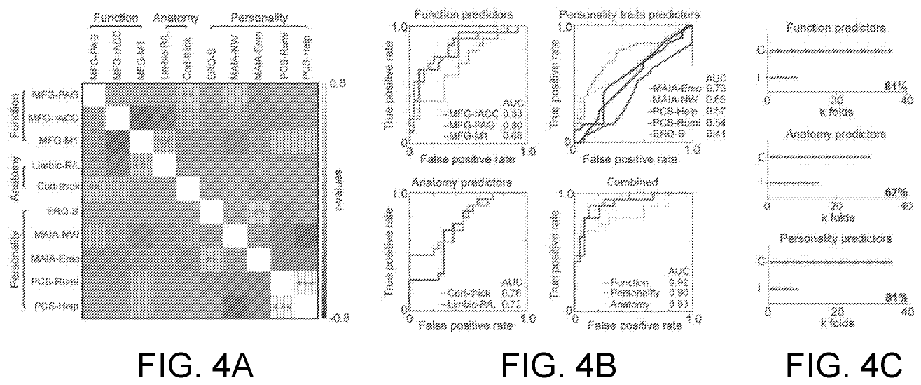

FIGS. 4A-4C depict a signature model predicting placebo propensity from brain and personality characteristics, using machine learning algorithms. FIG. 4A shows a correlation matrix with minimal correspondence between the brain parameters and psychological characteristics predisposing patients to placebo response. FIG. 4B shows receiver operating characteristic (ROC) curves computed for each functional predictor, each anatomical predictor, and each personality predictor using a support vector machine classifier (SVM). FIG. 4C illustrates results of a leave one subject out procedure used to test the accuracy of each model for predicting placebo response. MFG-PAG: middle frontal gyms-periaqueductal grey; MFG-rACC: middle frontal gyrus-rostral anterior cingulate cortex; MFG-M1: middle frontal gyrus-precentral gyrus; Limbic-R/L: limbic rightward asymmetry; Cort-thick: Cortical thickness in superior frontal gyrus; ERQ-s: Emotional Regulation suppression; MAIA-Emo: Multidimensional Assessment of Interoceptive Awareness emotional awareness; MAIA-NW: Multidimensional Assessment of Interoceptive Awareness not-worrying; PCS-Rumi: Pain Catastrophizing scale rumination; PCS-Help: Pain Catastrophizing scale helplessness). * p<0.05; ** p<0.01; *** p<0.001.

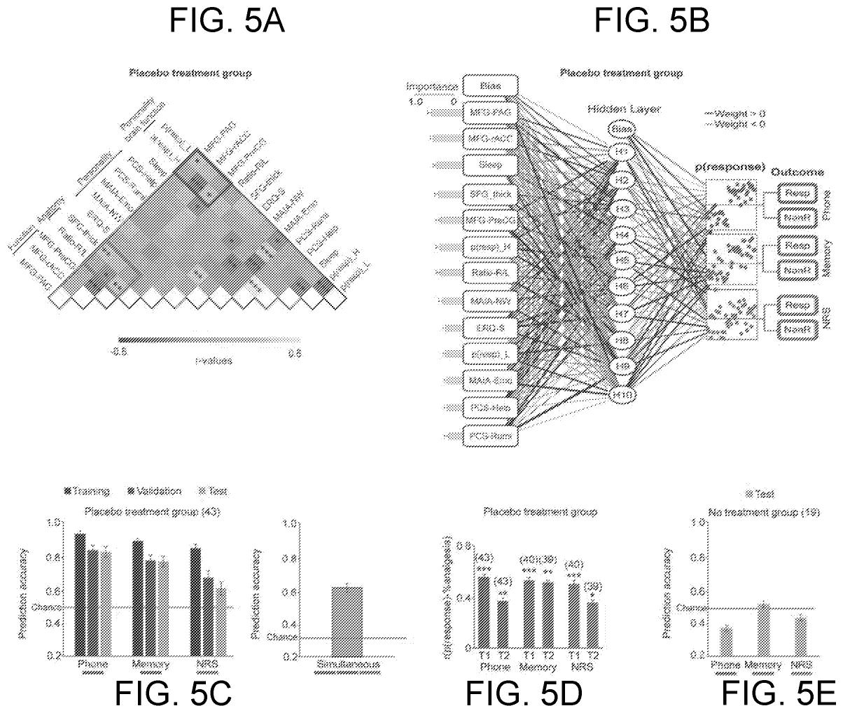

FIGS. 5A-5E depict a four-layered perceptron, using 13 inputs, which predicts future placebo pill response magnitude and categorical outcomes for 3 output measures and for 2 treatment timepoints. In FIG. 5A, a covariance matrix across all parameters predictive of placebo response shows relationships between: functional networks, anatomical predictors, sleep, personality traits, and components of the personality network. FIG. 5B shows that a 4-layered perceptron accurately predicts the likelihood of placebo pill response (3rd layer) and group outcomes (4th layer) when considering all three pain intensity outcomes (phone app, pain memory, and NRS). Importance scale depicts relative contribution of each input parameter based on sensitivity assessment in 100 simulations, for example. In FIG. 5C, grouping accuracy was determined by comparing the averaged prediction after training the perceptron model 100 times. Model accuracy is shown for each pain intensity outcome separately, and for all three simultaneously. FIG. 5D shows an averaged likelihood of response (3rd layer) correlated with the continuous variable % analgesia (100 simulations), for all pain outcomes at both treatment periods (T1, T2). FIG. 5E demonstrates that prediction accuracy was consistently above chance in all test groups of the placebo treatment group but failed to generalize to the no-treatment group. NI: normalized importance. * p<0.05; ** p<0.01; *** p<0.001. Error bars are 95% confidence interval in 100 simulations, for example.

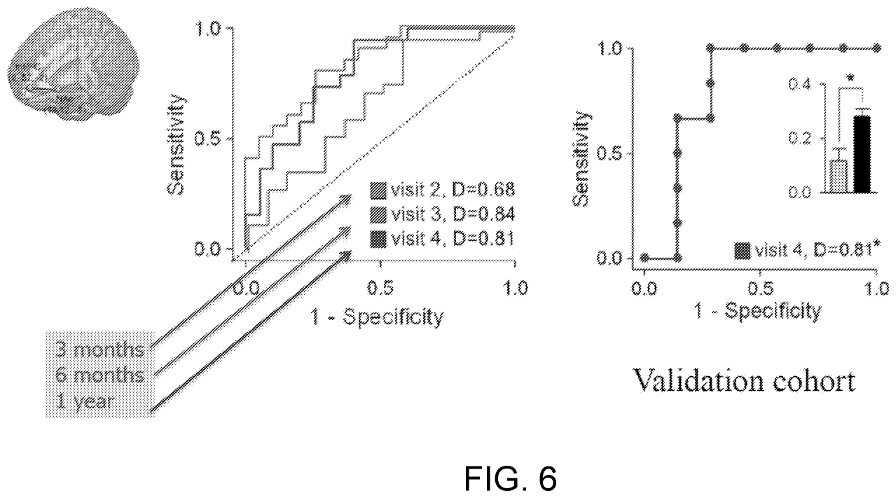

FIG. 6 shows an example extent of information shared between two brain regions, mPFC and NAc, in which a probability for risk of chronic pain in the future is calculated. ROC curves estimate this probability, for example.

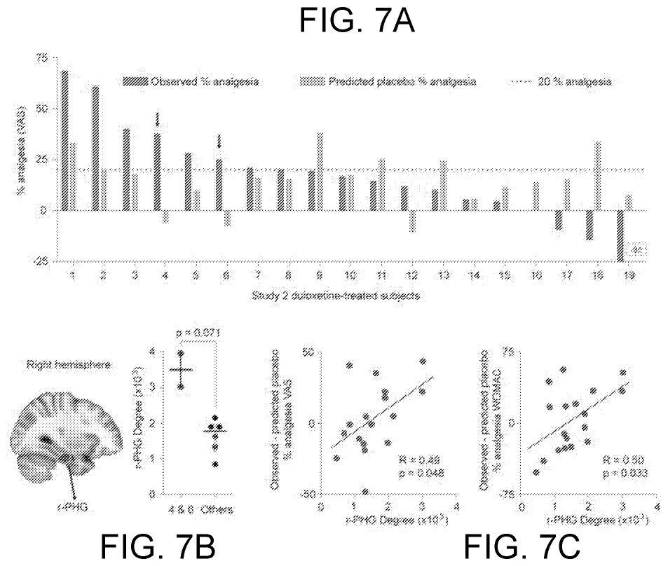

FIGS. 7A-7C show predictability of drug treatment response based on brain function. For example, right parahippocampal gyrus (r-PHG) degree counts predict future duloxetine response, based on modeling the placebo response in duloxetine-treated patients in study 2. In FIG. 7A, an empirical analgesia of individual duloxetine-treated patients (red) and the predicted placebo response (grey) are illustrated. The predicted placebo response was derived from the best-fit equation from study 1, which was applied to r-MFG degree count in duloxetine-treated patients. Patients with minimal predicted placebo and ≥20% empirical analgesia were considered mostly duloxetine responders (subjects 4 and 6; arrows). In FIG. 7B, contrasting the whole-brain degree counts of these two subjects with the six other duloxetine responders (subjects 1, 2, 3, 5, 7, and 8) revealed a right parahippocampal gyrus region (r-PHG) in which degree counts were higher in subjects 4 and 6 (scatter of individual values and median and quartiles are shown; Mann-Whitney U-test, p=0.071). In FIG. 7C, r-PHG degree count correlated with the difference between empirical analgesia and predicted placebo response for VAS (p=0.048) and WOMAC (p=0.033) outcomes, suggesting that the regional functional connections also reflect future placebo-corrected drug response for all 20 duloxetine-treated patients.

Thus, the figures provide evidence generated over many years regarding the feasibility of creating a general tool to optimize clinical care of chronic pain patients based on multi-dimensional machine learning modeling of data collected in a large cohort of patients. In certain examples, a tool is to be constructed, and data, collection, and output parameters generated. In certain examples, chronic pain patients can be tracked daily/weekly to determine main features in time and generate an algorithm to optimize/improve pain management recommendations that in time will significantly diminish all primary clinically impactful outcome features of participants, by generating individualized recommendations.

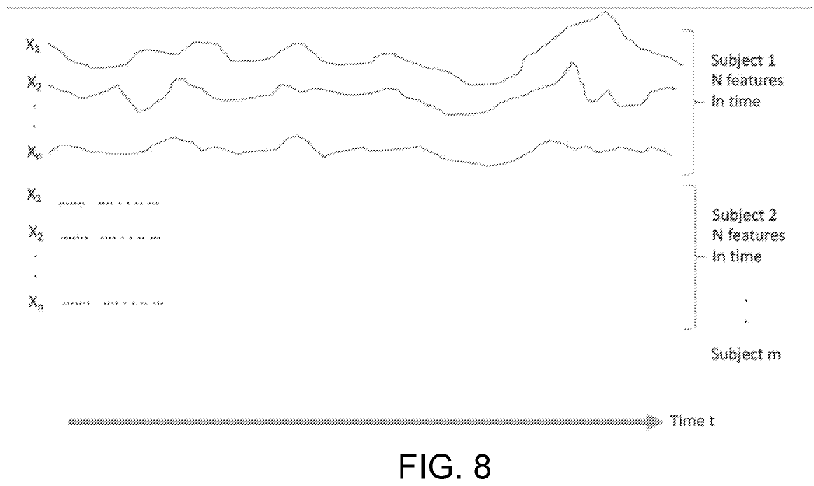

FIG. 8 shows an example smartphone application tracking individual subject features. Ongoing fluctuations of chronic pain related features (x1, . . . xn), in time, for a large number (m) of participants. Primary features include but are not limited to: pain level, mood, sleep, quality of life, mobility, etc. A primary objective is to decrease pain and improve quality of life, in time, and based on individualized recommendations made repeatedly in time (e.g., every ˜6-12 weeks) after monitoring features using recursive machine learning algorithms.

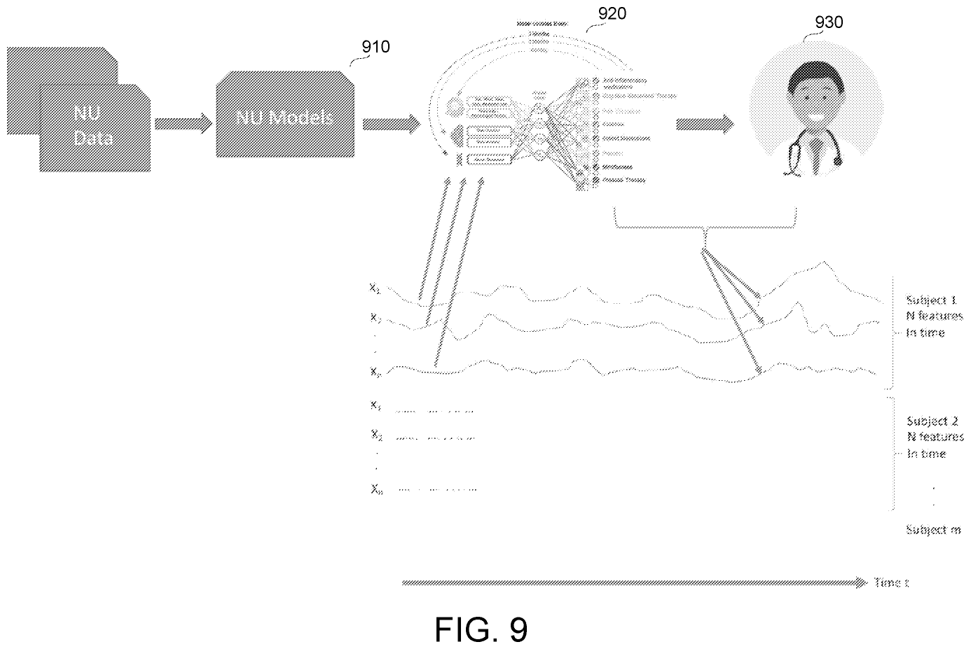

FIG. 9 shows an example fully integrated system 900 that incorporates models 910 with a recursive neural net 920, as well as with a clinical overview 930 of model output predictions. The system combines models 910 (e.g., based on ˜102 behavioral feature-based models; and on ˜103 brain imaging-based features; and only minimal genetic information, etc.), with the neural net 920, and with the ongoing pain related features, continuously collected in a large group of participants. The model outputs for any given subject are reviewed and partly corrected by clinical oversight 930. Eventually, the clinical oversight would be eliminated/minimized once the model becomes more stable and exhibits robust predictions. The model/clinical recommendation is passed to the participant and pain features rating and compliance with recommendation are tracked, recursively. In certain examples, the model is minimal but continues to learn and incorporate these memories within the network. In certain examples, the model can be enhanced with simulations as well clinical knowledge to enhance the neural net learning rate.

While example implementations are illustrated in conjunction with FIGS. 1-9, elements, processes and/or devices illustrated in conjunction with FIGS. 1-9 can be combined, divided, re-arranged, omitted, eliminated and/or implemented in any other way. Further, components disclosed and described herein can be implemented by hardware, machine readable instructions, software, firmware and/or any combination of hardware, machine readable instructions, software and/or firmware. Thus, for example, components disclosed and described herein can be implemented by analog and/or digital circuit(s), logic circuit(s), programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the components is/are hereby expressly defined to include a tangible computer readable storage device or storage disk such as a memory, a digital versatile disk (DVD), a compact disk (CD), a Blu-ray disk, etc. storing the software and/or firmware.

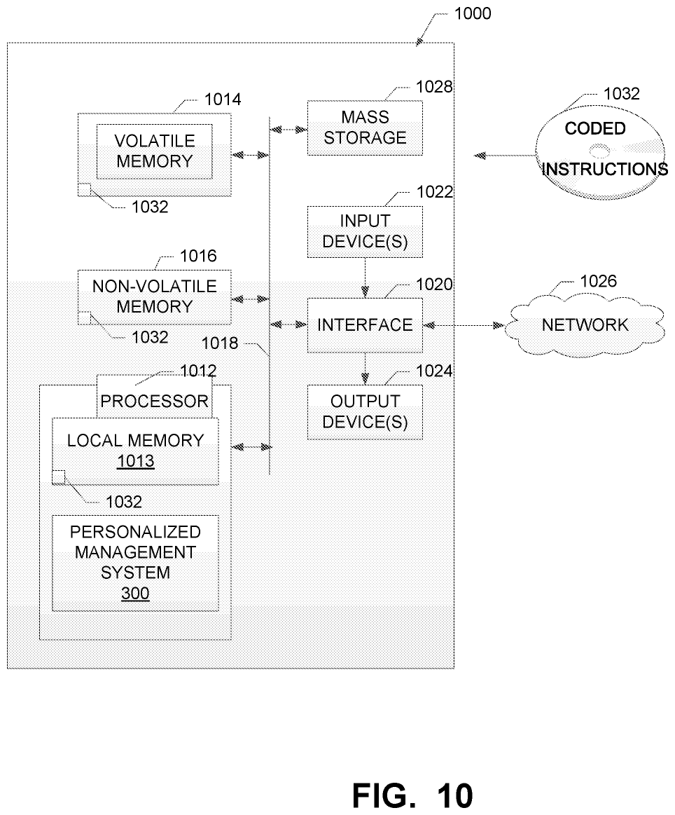

A flowchart representative of example machine readable instructions for implementing components disclosed and described herein is shown in conjunction with at least FIG. 1A. In the examples, the machine/computer readable instructions include a program for execution by a processor such as the processor 1012 shown in the example processor platform 600 discussed below in connection with FIG. 10. The program can be embodied in machine readable instructions stored on a tangible computer readable storage medium such as a CD-ROM, a floppy disk, a hard drive, a digital versatile disk (DVD), a Blu-ray disk, or a memory associated with the processor 1012, but the entire program and/or parts thereof could alternatively be executed by a device other than the processor 1012 and/or embodied in firmware or dedicated hardware. Further, although the example program is described with reference to the flowcharts illustrated in conjunction with at least FIG. 1A, many other methods of implementing the components disclosed and described herein can alternatively be used. For example, the order of execution of the blocks can be changed, and/or some of the blocks described can be changed, eliminated, or combined. Although the flowchart of at least FIG. 1A depicts example operations in an illustrated order, these operations are not exhaustive and are not limited to the illustrated order. In addition, various changes and modifications can be made by one skilled in the art within the spirit and scope of the disclosure. For example, blocks illustrated in the flowchart can be performed in an alternative order or can be performed in parallel.

As mentioned above, the example process(es) of at least FIG. 1A can be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example process(es) of at least FIG. 1A can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. In addition, the term “including” is open-ended in the same manner as the term “comprising” is open-ended.

FIG. 10 is a block diagram of an example processor platform 1000 structured to executing the instructions of at least FIG. 1A to implement the example components disclosed and described herein with respect to FIGS. 2-9. The processor platform 1000 can be, for example, a server, a personal computer, a mobile device (e.g., a cell phone, a smart phone, a tablet such as an iPad™), a personal digital assistant (PDA), an Internet appliance, or any other type of computing device.

The processor platform 1000 of the illustrated example includes a processor 1012. The processor 1012 of the illustrated example is hardware. For example, the processor 1012 can be implemented by integrated circuits, logic circuits, microprocessors or controllers from any desired family or manufacturer.

The processor 1012 of the illustrated example includes a local memory 1013 (e.g., a cache). The example processor 1012 of FIG. 10 executes the instructions of at least FIG. 1A to implement the systems and infrastructure and associated methods of FIGS. 2-9 such as the example data-driven personalized management system 300, etc. The processor 1012 of the illustrated example is in communication with a main memory including a volatile memory 1014 and a non-volatile memory 1016 via a bus 1018. The volatile memory 1014 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 1016 can be implemented by flash memory and/or any other desired type of memory device. Access to the main memory 1014, 1016 is controlled by a clock controller.

The processor platform 1000 of the illustrated example also includes an interface circuit 1020. The interface circuit 1020 can be implemented by any type of interface standard, such as an Ethernet interface, a universal serial bus (USB), and/or a PCI express interface.

In the illustrated example, one or more input devices 1022 are connected to the interface circuit 1020. The input device(s) 1022 permit(s) a user to enter data and commands into the processor 1012. The input device(s) can be implemented by, for example, a sensor, a microphone, a camera (still or video), a keyboard, a button, a mouse, a touchscreen, a track-pad, a trackball, isopoint and/or a voice recognition system.

One or more output devices 1024 are also connected to the interface circuit 1020 of the illustrated example. The output devices 1024 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 1020 of the illustrated example, thus, typically includes a graphics driver card, a graphics driver chip or a graphics driver processor.

The interface circuit 1020 of the illustrated example also includes a communication device such as a transmitter, a receiver, a transceiver, a modem and/or network interface card to facilitate exchange of data with external machines (e.g., computing devices of any kind) via a network 1026 (e.g., an Ethernet connection, a digital subscriber line (DSL), a telephone line, coaxial cable, a cellular telephone system, etc.).

The processor platform 1000 of the illustrated example also includes one or more mass storage devices 1028 for storing software and/or data. Examples of such mass storage devices 1028 include floppy disk drives, hard drive disks, compact disk drives, Blu-ray disk drives, RAID systems, and digital versatile disk (DVD) drives.

The coded instructions 1032 of FIG. 10 can be stored in the mass storage device 1028, in the volatile memory 1014, in the non-volatile memory 1016, and/or on a removable tangible computer readable storage medium such as a CD or DVD.

From the foregoing, it will be appreciated that the above disclosed methods, apparatus, and articles of manufacture have been disclosed to improve the functioning of a computer and/or computing device and its interaction with image and other patient data for patient classification and personalized medicine.

Certain examples facilitate monitoring of subjects over time using clinical oversight and applying machine learning to understand patient conditions, categorize patients/conditions, and treat patient conditions. In certain examples, adaptive deep learning packages can evaluate data and change model weight over time based on feedback and learning. Machine learning tools (e.g., a recursive neural network, etc.) can be used for clinical decision making based on continuous monitoring of patient image and personality data, for example. Monitoring can be in time, monitoring a large group of subjects and identifying optimum treatment paths for individual subjects in the group, for example. A process of inputs can be defined and correlated to a model and outputs.

Such models are important for chronic pain because no pathology is currently identifiable that relates to chronic pain. A pain property/personality/psychology determines a chronic pain state, and understanding applicable types/categories is a best way to identify a best treatment protocol for the best outcome for an individual patient. Using a database and characterization of time-varying pain, along with personality changes and impact of environment and drugs, etc., a future can be predicted for a patient based on current and future treatment options. Recommendation(s) can be provided (e.g., via a smartphone app, etc.) based on a categorization of a person.

In certain examples, a set of questions (e.g., 50 questions, 100 questions, 300 questions, etc.) can be determined and scored according to a scheme to stratify patients according to category. A subset of questions can be identified to categorize a patient.

One or more aspects discussed herein can be embodied in computer-usable or readable data and/or computer-executable instructions, such as in one or more program modules, executed by one or more computers or other devices as described herein. Generally, program modules include routines, programs, objects, components, data structures, and the like. that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various embodiments. In addition, the functionality can be embodied in whole or in part in firmware or hardware equivalents such as integrated circuits, field programmable gate arrays (FPGA), and the like. Particular data structures can be used to more effectively implement one or more aspects discussed herein, and such data structures are contemplated within the scope of computer executable instructions and computer-usable data described herein. Various aspects discussed herein can be embodied as a method, a computing device, a system, and/or a computer program product.

Although the present invention has been described in certain specific aspects, many additional modifications and variations would be apparent to those skilled in the art. In particular, any of the various processes described above can be performed in alternative sequences and/or in parallel (on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method, comprising:
obtaining, by a processor platform, patient data indicating a placebo propensity for a patient;
calculating, by the processor platform, predicted response outcomes for the patient based on the placebo propensity;
calculating, by the processor platform, a predicted risk of chronic pain for the patient based on the patient data;
calculating, by the processor platform, a predicted drug treatment response for the patient based on the patient data;
obtaining, by the processor platform, revised patient data indicating chronic condition parameters for the patient;
generating, by the processor platform, a treatment plan for the patient based on the predicted response outcomes, the predicted risk of chronic pain, the predicted drug treatment response, and the revised patient data; and
administering the treatment plan to the patient.

2. The method of claim 1, wherein the placebo propensity is determined based on brain characteristics and personality characteristics.

3. The method of claim 1, wherein the chronic condition parameters are selected from the group consisting of pain level, mood, sleep, quality of life, and mobility.

4. The method of claim 1, wherein the predicted drug treatment response is calculated based on brain anatomy of the patient, functional connectivity of the patient, and gene expression identifiers of the patient.

5. The method of claim 1, wherein the predicted response outcomes are calculated using a machine classifier trained using a set of historical data indicating functional networks, anatomical predictors, sleep data, and personality traits.

6. The method of claim 1, wherein the predicted risk of chronic pain is calculated based on a receiver operating characteristic generated based on brain region information sharing for the patient.

7. The method of claim 1, wherein the predicted drug treatment response is calculated based on a patient response to an administration of a particular drug.

8. A processing platform, comprising:
a processor; and
a memory storing instructions that, when executed by the processor, cause the processing platform to:
obtain patient data indicating a placebo propensity for a patient;
calculate predicted response outcomes for the patient based on the placebo propensity;
calculate a predicted risk of chronic pain for the patient based on the patient data;
calculate a predicted drug treatment response for the patient based on the patient data;
obtain revised patient data indicating chronic condition parameters for the patient;
generate a treatment plan for the patient based on the predicted response outcomes, the predicted risk of chronic pain, the predicted drug treatment response, and the revised patient data; and
administer the treatment plan to the patient.

9. The processing platform of claim 8, wherein the placebo propensity is determined based on brain characteristics and personality characteristics.

10. The processing platform of claim 8, wherein the chronic condition parameters are selected from the group consisting of pain level, mood, sleep, quality of life, and mobility.

11. The processing platform of claim 8, wherein the predicted drug treatment response is calculated based on brain anatomy of the patient, functional connectivity of the patient, and gene expression identifiers of the patient.

12. The processing platform of claim 8, wherein the predicted response outcomes are calculated using a machine classifier trained using a set of historical data indicating functional networks, anatomical predictors, sleep data, and personality traits.

13. The processing platform of claim 8, wherein the predicted risk of chronic pain is calculated based on a receiver operating characteristic generated based on brain region information sharing for the patient.

14. The processing platform of claim 8, wherein the predicted drug treatment response is calculated based on a patient response to an administration of a particular drug.

15. A non-transitory machine-readable medium storing instructions that, when executed by one or more processors, cause the one or more processors to perform steps comprising:
obtaining patient data indicating a placebo propensity for a patient;
calculating predicted response outcomes for the patient based on the placebo propensity;
calculating a predicted risk of chronic pain for the patient based on the patient data;
calculating a predicted drug treatment response for the patient based on the patient data;
obtaining revised patient data indicating chronic condition parameters for the patient;
generating a treatment plan for the patient based on the predicted response outcomes, the predicted risk of chronic pain, the predicted drug treatment response, and the revised patient data; and
administering the treatment plan to the patient.

16. The non-transitory machine-readable medium of claim 15, wherein the placebo propensity is determined based on brain characteristics and personality characteristics.

17. The non-transitory machine-readable medium of claim 15, wherein the chronic condition parameters are selected from the group consisting of pain level, mood, sleep, quality of life, and mobility.

18. The non-transitory machine-readable medium of claim 15, wherein the predicted drug treatment response is calculated based on brain anatomy of the patient, functional connectivity of the patient, and gene expression identifiers of the patient.

19. The non-transitory machine-readable medium of claim 15, wherein the predicted response outcomes are calculated using a machine classifier trained using a set of historical data indicating functional networks, anatomical predictors, sleep data, and personality traits.

20. The non-transitory machine-readable medium of claim 15, wherein the predicted drug treatment response is calculated based on a patient response to an administration of a particular drug.
